(19) United States
(12) Patent Application Publication
(10) Pub. No.: US 2006/0258945 A1
(43) Pub. Date: Nov. 16, 2006

(54) Mounting structure with band fixing bar and detecting apparatus

(75) Inventors: Katsuyoshi Nishii, Okazaki-city (JP); Teiyu Kimura, Nagoya-city (JP); Kazuhiro Sakai, Anjo-city (JP); Fumiya Nagai, Anjo-city (JP); Miwaji Ikeya, Anjo-city (JP); Satoshi Nozoe, Kariya-city (JP)

Correspondence Address: POSZ LAW GROUP, PLC, 12040 SOUTH LAKES DRIVE, SUITE 101, RESTON, VA, 20191, US

(73) Assignee: DENSO CORPORATION, Kariya-city (JP)

(21) Appl. No.: 11/417,111

(22) Filed: May 4, 2006

(30) Foreign Application Priority Data
May 16, 2005 (JP) 2005-142939

(51) Int. Cl. A61B 5/02; A61B 5/04

(52) U.S. Cl. 600/500; 600/390

(57) ABSTRACT

A detecting apparatus for detecting a living body information includes a sensor body provided to contact a surface of a living body, and a band fixing bar elongated in a longitudinal direction, through which a band is engaged and is detachably connectable to the sensor body. In the detecting apparatus, the sensor body has a pair of holding portions for holding end portions of the band fixing bar in the longitudinal direction. The holding portion has an inlet concave portion from which the end portion of the band fixing bar is inserted, and a recess portion for receiving the end portion of the band fixing bar. Furthermore, the recess portion communicates with the inlet concave portion and recesses toward the living body, and the end portion of the band fixing bar is fitted into the recess portion. Therefore, the band fixing bar does not easily come off from the sensor body.

### CROSS REFERENCE TO RELATED APPLICATION

This application is based on Japanese Patent Application No. 2005-142939 filed on May 16, 2005, the contents of which are incorporated herein by reference in its entirety.

### FIELD OF THE INVENTION

The present invention relates to a mounting structure with a band fixing bar. More particularly, the present invention relates to a band fixing bar mounting mechanism and an apparatus using the same, which can be suitably used for a pulse wave sensor for detecting pulse waves, for example.

### BACKGROUND OF THE INVENTION

A conventional wrist watch type pulse wave sensor for detecting a pulse rate by using a reflective optical apparatus has been known (JP-A-2001-78972). In the pulse wave sensor, a wrist band is attached to a sensor body having the reflective optical apparatus therein. The sensor body is fitted to a wrist by winding the band around the wrist and making the band stay around the wrist with a hook.

However, in the above-described structure, the band is fixed to the sensor body by using a spring bar bent in an axial direction thereof, which is manufactured for wrist watches. Therefore, an attachment/detachment of the wrist band to/from the sensor body is difficult, especially at the time of detachment.

Furthermore, since a structure of the spring bar is complicated, it has pushed up manufacturing cost thereof.

### SUMMARY OF THE INVENTION

In view of the above-described problems, it is an object of the present invention to provide a mounting structure with a band fixing bar, which can prevent the band fixing bar from being easily removed after an attachment while a band can be easily attached to or detached from a sensor body.

It is another object of the present invention to provide a detecting apparatus for detecting a living body information with the mounting structure.

According to an aspect of the present invention, a mounting structure includes an apparatus held by a band to contact a surface of a living body, a band fixing bar elongated in a longitudinal direction, through which the band is engaged and is detachably connectable to the apparatus, and a pair of holding portions provided in the apparatus, for holding end portions of the band fixing bar in the longitudinal direction. The holding portion has an inlet concave portion from which the end portion of the band fixing bar is inserted, and a recess portion for receiving the end portion of the band fixing bar. In the mounting structure, the recess portion communicates with the inlet concave portion and recesses from an end of the inlet concave portion toward the living body, and the end portion of the band fixing bar is fitted into the recess portion. Accordingly, the band fixing bar does not easily come off from the apparatus, when the apparatus is attached to a human.

The band fixing bar can have an elasticity to be bendable in a radial direction approximately perpendicular to the longitudinal direction. In this case, a protruding portion can be located between the holding portions in the axial direction, and the protruding portion protrudes from the apparatus to the living body at an outer position of the band fixing bar in the radial direction so as to prevent the band fixing bar from falling. In this case, it can further prevent the band fixing bar from being removed. The protruding portion can be provided with a lowest point on a side of the living body to contact the band fixing bar when the band fixing bar is inserted into the inlet concave portion. For example, the protruding portion can be provided approximately in a middle area of the holding portions, or can be constructed with a plurality of convex portions separate from each other in the axial direction of the band fixing bar.

Furthermore, the recess portion of the apparatus can be set at a lowest point on a side of the living body, and the lowest point can be made lower than a lower end of the inlet concave portion in a state where the apparatus is positioned above the surface of the living body. In this case, the band fixing bar fitted into the recess portion is not easily come off from the inlet concave portion. In addition, a maximum width between the inlet concave portions of the holding portions can be set greater than a dimension of the band fixing bar in the longitudinal direction.

The holding portions can be provided at an inner side from an outer periphery of the apparatus in the longitudinal direction. Furthermore, the holding portions can be provided so that the band fixing bar mounted to the holding portions is covered by the apparatus when viewed from an opposite direction with respect to the living body.

Furthermore, a trench portion recessed from an outer wall of the apparatus can be provided so that a bar part of the band fixing bar between the end portions is located in the trench portion. For example, the band fixing bar has a shape of a circular cylinder, a rectangular prism or a hexagonal prism elongated in the longitudinal direction.

According to another aspect of the present invention, the band fixing bar can be provided with at least one convex portion protruding from one end surface of the band fixing bar in the longitudinal direction. In this case, a thickness of the convex portion is made smaller than a thickness of the band fixing bar, the inlet concave portion has an open shape corresponding to a sectional shape of the convex portion with the thickness, and the convex portion is provided to pass through the inlet concave portion in a predetermined direction. Accordingly, the band can be easily attached to or detached from the apparatus, while it can prevent the band fixing bar from removing when using the apparatus.

The mounting structure can be suitably used for a detecting apparatus for detecting a living body information. For example, the band has a loop at each end and the band fixing bars are inserted into the loops. By connecting the band fixing bars having the band to a sensor body, the band is attached to the sensor body. Therefore, the bar hardly comes off from sensor body once mounted. A measurement part of the sensor body can detect human body information by winding the band around an arm or a wrist.

### BRIEF DESCRIPTION OF THE DRAWINGS

The above and other objects, features and advantages of the present invention will become more apparent from the following detailed description of preferred embodiments made with reference to the accompanying drawings, in which:

FIG. 1 is a schematic diagram illustrating a pulse wave sensor in a first embodiment of the present invention;

FIGS. 2A to 2C are perspective views of bars which can be used as a band fixing bar;

FIG. 3 is a schematic diagram illustrating an elasticity of the band fixing bar;

FIG. 4 is a schematic sectional view illustrating a band in its straightened state;

FIGS. 5A and 5B are side views of a sensor body of the sensor in FIG. 1 viewed from an arrow A;

FIG. 6A is a schematic cross-sectional view of the sensor body taken along the line B-B in FIG. 5A, and FIG. 6B is a schematic diagram illustrating a vicinity of a holding portion in FIG. 5A;

FIGS. 7A and 7B are cross-sectional views of the sensor body taken along the line B-B in FIG. 5A;

FIG. 8 is a schematic diagram illustrating another example in which plural convex portions are used;

FIG. 9A is a perspective view of a band fixing bar used for a pulse wave sensor in a second embodiment of the present invention, and FIG. 9B is a schematic diagram illustrating a vicinity of a mounting mechanism of a sensor body when viewed from an axial direction of the band fixing bar; and

FIG. 10 A is a side view of a sensor body in a third embodiment of the present invention, and FIG. 10B is a schematic diagram showing a relation of a band fixing bar and a band in FIG. 10A.

### DETAILED DESCRIPTION OF PREFERRED EMBODIMENTS

Embodiments of the present invention will be described with reference to accompanying drawings.

### First Embodiment

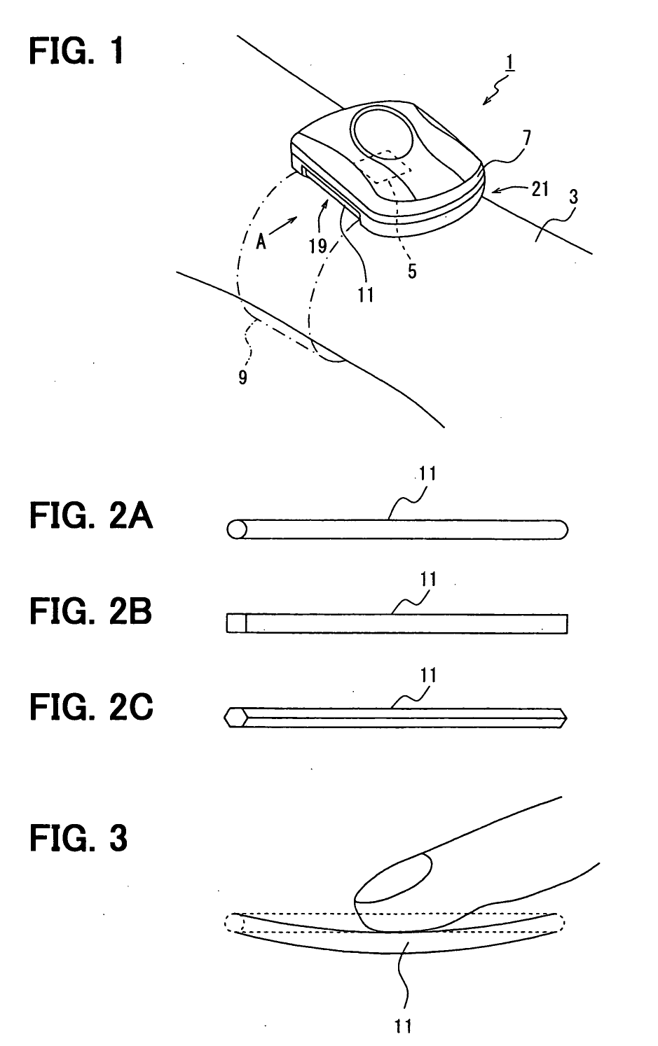

A living body information detecting apparatus, e.g., a pulse wave sensor, for optically detecting a pulse wave by attaching the apparatus closely to an arm of a human body will be described as an example. A basic structure of the pulse wave sensor will be described referring to FIG. 1 through FIG. 4

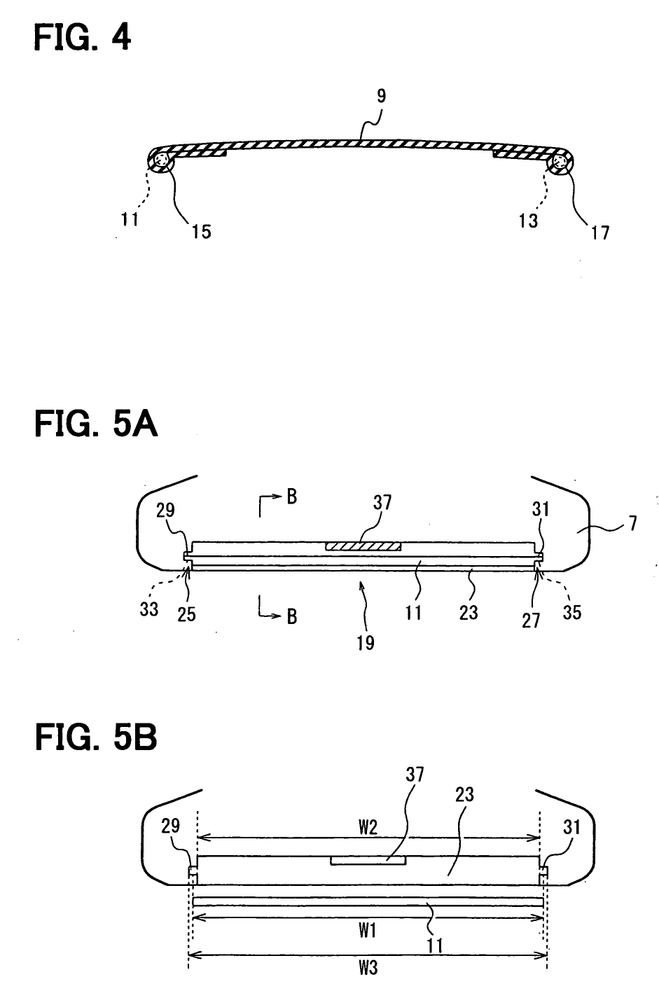

A pulse wave sensor 1 is a portable apparatus, which can be attached to e.g., a human arm 3 as shown in FIG. 1. The pulse wave sensor 1 includes a sensor body 7 having a measurement part 5 that measures a pulse wave optically, a strip-shaped band 9 for attaching the sensor body 7 to the arm 3, and band fixing bars 11, 13 (shown in FIG. 4) for attaching the band 9 to the sensor body 7.

Although not shown, the measurement part 5 is constructed of a green light-emitting diode (a green LED), an infrared-emitting diode (an IRED) and a photodiode (a PDIO). The PDIO receives reflected lights of the green LED and IRED. The green LED is for detecting the pulse wave by emitting the green light to the human body and detecting changes in an amount of hemoglobin of blood capillaries from its reflected light. The IRED is for detecting body movement by emitting the infrared light to the human body and detecting the body movement from its reflected light.

The sensor body 7 is an approximately rectangular and plate-shaped apparatus having an indicator and switches (neither shown) on an upper surface thereof. The measurement part 5 is provided on a back surface of the sensor body 7, which closely contacts a surface of the arm 3.

The band fixing bar 11 is, for example, a cylinder with diameter of 2 mm and length 33 mm as shown in FIG. 2A. The diameter of the bar 11 is uniform in an axial direction thereof. The band fixing bar 11 is made of resinous material like an acrylic resin. As shown in FIG. 3, when pushed with a finger, the bar 11 bends in the radial direction thereof (perpendicular with respect to the axial direction of the bar 11) and bends back to a normal state when the pressure thereon is removed.

The shape of the band fixing bar 11 can be a rectangular prism as shown in FIG. 2B or a hexagonal prism as shown in FIG. 2C. The material is not necessarily limited to the acrylic resin but can be other resinous materials such as polycarbonate. Metals can be used for the bar likewise. It is more cost effective if a singular material is used for the band fixing bar 11.

The band 9 is made of rubber or circular-knitted fabric having an elasticity. The ends of the band 9 are made into loops having through holes 15, 17 for receiving the band fixing bars 11, 13. The band fixing bars 11, 13 are inserted into the through holes 15, 17 of the band 9, respectively.

Therefore, by attaching the band fixing bars 11, 13 to mounting mechanisms 19, 21 provided at two ends of the sensor body 7, the band 9 can be attached to the sensor body 7.

The band 9 and the sensor body 7 form a ring shape so that the arm 3 can be put through the ring. The pulse wave sensor 1 can be fitted to the arm 3 after being attached, since the band 9 has the elasticity.

Next, a structure and a method of mounting the band fixing bars 11,13 to the sensor body 7 will be described referring to FIG. 5A through FIG. 7B.

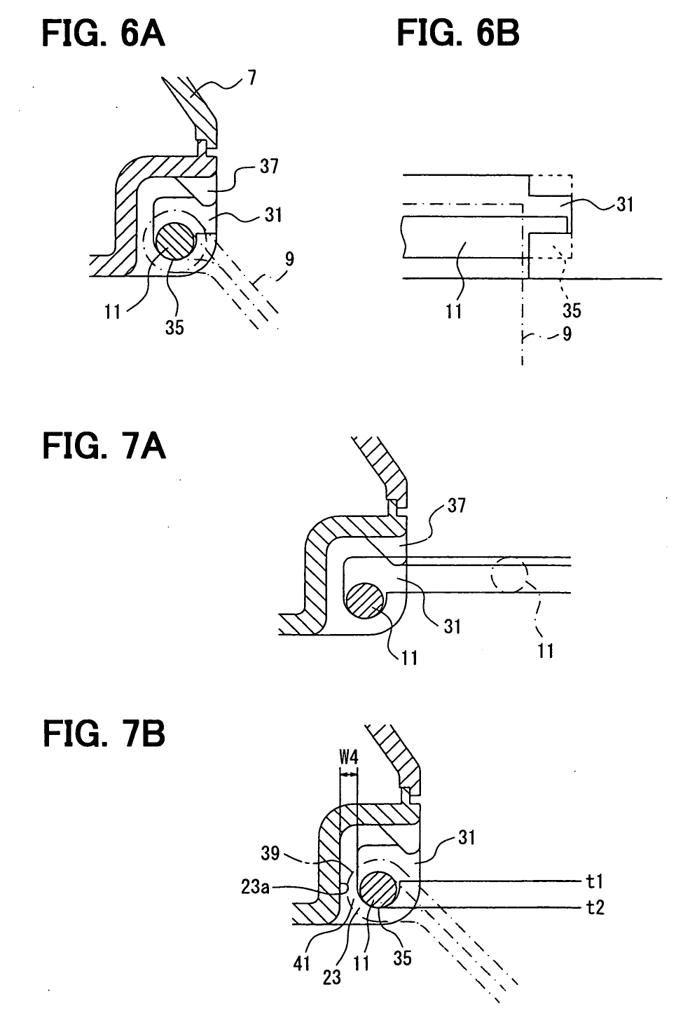

FIGS. 5A and 5B are side views of the sensor body 7 viewed from an arrow A in FIG. 1. FIGS. 6A, 7A and 7B are cross-sectional views of the mounting mechanism of the sensor body 7 taken along the line B-B in FIG. 5A. As shown in FIG. 5A, the mounting mechanism 19 for receiving the band fixing bar 11 is provided at a down corner of the sensor body 7, which is at a side of the sensor body 7 facing the living body (i.e., arm 3). Here, the mounting mechanism 21 has the same structure as in the mechanism 19, the following explanation will be given to the mechanism 19.

The mounting mechanism 19 has a long trench portion 23 that is provided in a center area on a side back surface of the mechanism 19 in a left-right direction of FIG. 5A. Holding portions 25, 27 for holding the band fixing bar 11 are provided, respectively, at ends of the trench portion 23. The left-right direction corresponds to the axial direction of the band fixing bar 11 when attached to the mounting mechanism 19.

Inlet concave portions 29, 31, each of which has a rectangular shape of 2.1 mm long and 2 mm and opens towards front in FIG. 5A, are provided at the holding portions 25, 27. The left and right ends of the band fixing bar 11 fit respectively into the inlet concave portions 29, 31.

A length W2 of the trench portion 23 in the left-right direction is set as 30 mm, for example, which is shorter than a length W1 of the band fixing bar 11 as shown in FIG. 5B. A length W3, which is a dimension including the length of the trench portion 23 and the inlet concave portions 29, 31, is set as 34 mm for example, to be longer than the length W1 of the band fixing bar 11.

Notched recess portions 33, 35 shown in FIG. 5A are provided further inward from the inlet concave portions 29, 31 to a side of a center part of the sensor body 7. The notched recess portions 33, 35 are provided in the sensor body 7 to the side of the arm (in the lower direction of FIG. 6A). Therefore, the both ends of the band fixing bar 11 inserted from the inlet concave portions 29,31 fall into the notched recess portions 33, 35 to be held therein as shown in FIG. 6A and FIG. 6B.

As shown in FIGS. 5A, 5B, a convex portion 37 (indicated with diagonal lines in FIG. 5A) for preventing the band fixing bar 11 from coming off from the mechanism 19, is provided at a middle of the trench portion 23 in the left-right direction. The convex portion 37 protrudes downward from a top surface above the trench portion 23 to have a protruding part protruding into the trench portion 23. As shown in FIGS. 5A, 5B, the protruding part has substantially a rectangular section.

As shown in FIG. 7A, a lower end (a lowest point) of the convex portion 37 is set to be lower than an upper end of the band fixing bar 11 when the bar 11 is to be fitted into the inlet concave portions 29, 31. Therefore, once the bar 11 is fitted into the inlet concave portions 29, 31 by passing the convex portion 37, the bar 11 does not easily come off from the inlet concave portions 29,31 to an outside.

A lowest point t2 of the recess portions 33,35 is set lower than a lowest point t1 of the inlet concave portions 29,31, as shown in FIG. 7B. The lowest point t1 is also set higher than an axial center point of the band fixing bar 11 fitted into the inlet concave portions 29, 31.

The inlet concave portions 29,31 as well as an inner circumferential surface of the recess portions 33,35 at a farther inner area therefrom are formed higher than the center trench portion 23 so that a stepping portion 39 is formed therebetween. A space 41 having a width W4 is formed between a wall surface 23a of the center trench portion 23 and the band fixing bar 11. The band 9 is fitted to the band fixing bar 11 by making use of the space 41.

In this embodiment, the band fixing bar 11 is bendable in the radial direction so as to pass the convex portion 37 and to be inserted into the left and right inlet concave portions 29,31. As a result, the bar 11 is fitted into the left and right recess portions 33, 35. Therefore, the bar 11 is attached to the holding portions 25, 27 thereby the band 9 can be attached to the sensor body 7.

According to the first embodiment of the present invention, the attachment and removal of the band 9 becomes easy. Since the structure of the band fixing bar 11 is simple, a production cost thereof can be kept low.

Also, the lower end of the convex portion 37 is set to become lower than the upper end of the band fixing bar 11 (the upper end of an opening of the inlet concave portions 29, 31) when the bar 11 is fitted into the inlet concave portions 29, 31. Therefore, if the band fixing bar 11 is pulled toward an outer direction (e.g., toward the inlet concave portions 29, 31), the band fixing bar 11 hits against the convex portion 37. Thus, the bar 11 hardly comes off to the outside. In addition, because the ends of the band fixing bar 11 are fitted into the recess portions 33, 35, the bar 11 does not easily come off to the outside.

Also, in this embodiment, the holding portions 25, 27 do not protrude from the sensor body 7 to the outer direction. The holding portions 25, 27, the recess portions 33, 35 and the band fixing bar 11 are all located inside the outer periphery of the sensor body 7. Therefore, when viewed from the outside of the sensor body 7, an exterior looks smooth, since there is few irregularities found on the surface of the sensor body 7. Also, this structure hardly produces a space between the sensor body 7 and the arm 3, the sensor body 7 fits closely to the surface of the arm 3. Therefore, an accuracy in measurement by an optical means also improves.

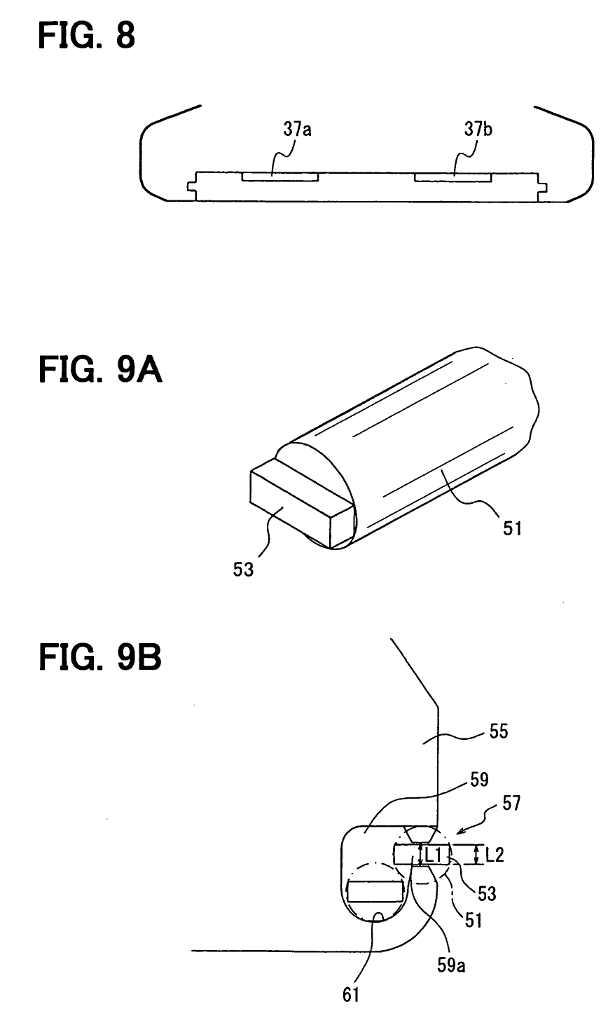

As shown in FIG. 8, a plurality of convex portions 37a, 37b for preventing the band fixing bar 11 from coming off from the mechanism 19 can be provided above the trench portion 23 side by side in the left-right direction. In this case, when the both ends of the band fixing bar 11 are fitted into the left and right inlet concave portions 29,31, the band 11 needs to be more bent in the radial direction when the band fixing bar 11 is fitted into the inlet concave portions 29,31 by passing the single convex portion 37. Therefore, when the band fixing bar 11 is pulled outwardly (e.g., to the side of the inlet concave portions 29,31), the bar 11 is more effectively prevented from falling out from the inlet concave portions 29,31.

### Second Embodiment

Next, the second embodiment according to the present invention will be described with reference to FIGS. 9A and 9B. Explanations on the same components as in the first embodiment will be omitted. In this embodiment, a pulse wave sensor has substantially cuboid plate like convex portions 53 at both ends of a cylindrical band fixing bar 51 in the axial direction thereof, as shown in FIG. 9A.

A sensor body 55 has a mounting mechanism 57 and an inlet concave portion 59 is provided in the mechanism 57. A dimension of an opening portion 59a of the inlet concave portion 59 is formed smaller than a diameter of a cylindrical part of the band fixing bar 51.

To be specific, a dimension L2, which is a thickness of the convex portion 53, is so set that the convex portion 53 can be inserted along its longitudinal direction, when the band fixing bar 51 is fitted into the inlet concave portion 59.

An up-down dimension L1 of the opening portion 59a is set almost same as the dimension L2 or slightly larger than the dimension L1.

Therefore, once the band fixing bar 51 passes through the opening portion 59a of the inlet concave portion 59 and is received by a recess 61, the bar 51 does not come off to an outside easily even when the bar 51 is pulled towards the outside. In the second embodiment, the other parts can be made similar to the above-described first embodiment.

### Third Embodiment

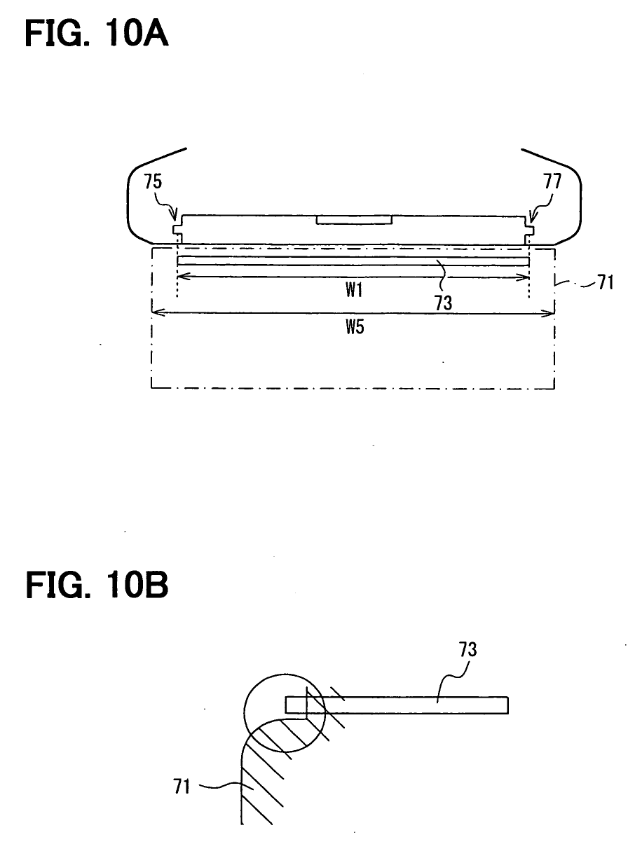

The third embodiment according to the present invention will be described. Explanations on the same components as in the first embodiment will be omitted. As shown in FIG. 10A, when an elastic material is used for a band 71, the band 71 having a width W5, which is greater than a width W1 of a band fixing bar 73 in an axial direction, can be used.

Also, as schematically shown in FIG. 10B, the width of the band 71 in a vicinity of the bar 73 is made narrower so that the band fixing bar 73 can be easily fitted into holding portions 75, 77.

The larger the width of the band 71 becomes, the more stable the band 71 stays around an arm when it is worn. As a result, an accuracy in measurement also improves.

While the invention has been described with reference to preferred embodiments thereof, it is to be understood that the invention is not limited to the preferred embodiments and constructions.

For example, in the above described embodiments, the mounting mechanism of the sensor body has both the convex portions for preventing the fixing bar from coming off and the recess portions for receiving the bar. However, by providing either the convex portions or the recess portions, the mechanism still has enough holding effect. In case only the recess portions are provided, the band fixing bar having non-elasticity can be used.

Also, when non-elastic material is used for the band, a length of the band is preferably adjusted in accordance with a dimension of an arm.

In the third embodiment, the other parts can be made similar to those of the first or second embodiment.

The invention is intended to cover various modification and equivalent arrangements. In addition, while the various elements of the preferred embodiments are shown in various combinations and configurations, which are preferred, other combinations and configuration, including more, less or only a single element, are also within the spirit and scope of the invention.

What is claimed is:

1. A mounting structure comprising:
an apparatus held by a band to contact a surface of a living body;
a band fixing bar elongated in a longitudinal direction, through which the band is engaged and is detachably connectable to the apparatus; and
a pair of holding portions provided in the apparatus, for holding end portions of the band fixing bar in the longitudinal direction, wherein:
the holding portion has an inlet concave portion from which the end portion of the band fixing bar is inserted, and a recess portion for receiving the end portion of the band fixing bar;
the recess portion communicates with the inlet concave portion and recesses from an end of the inlet concave portion toward the living body; and
the end portion of the band fixing bar is fitted into the recess portion.

2. A mounting structure comprising:
an apparatus held by a band to contact a surface of a living body;
a band fixing bar having an elasticity to be bendable in a radial direction and detachably connectable to the apparatus; and
a pair of holding portions provided in the apparatus, for holding end portions of the band fixing bar in an axial direction perpendicular to the radial direction, wherein the holding portion has an inlet concave portion from which the end portion of the band fixing bar is inserted; and
a protruding portion located between the holding portions in the axial direction, wherein the protruding portion protrudes from the apparatus toward the living body at an outer position of the band fixing bar in the radial direction so as to prevent the band fixing bar from falling.

3. The mounting structure according to claim 1, wherein the band fixing bar has an elasticity to be bendable in a radial direction approximately perpendicular to the longitudinal direction, the mounting structure further comprising
a protruding portion located between the holding portions in the axial direction, wherein the protruding portion protrudes from the apparatus to the living body at an outer position of the band fixing bar in the radial direction so as to prevent the band fixing bar from falling.

4. The mounting structure according to claim 1, wherein the recess portion of the apparatus has a lowest point on a side of the living body, the lowest point being lower than a lower end of the inlet concave portion in a state where the apparatus is positioned above the surface of the living body.

5. The mounting structure according to claim 2, wherein the protruding portion has a lowest point on a side of the living body to contact the band fixing bar when the band fixing bar is inserted into the inlet concave portion.

6. The mounting structure according to claim 1, wherein the holding portions are provided at an inner side from an outer periphery of the apparatus in the longitudinal direction.

7. The mounting structure according to claim 1, wherein the holding portions are provided so that the band fixing bar mounted to the holding portions is covered by the apparatus when viewed from an opposite direction with respect to the living body.

8. The mounting structure according to claim 1, wherein the band fixing bar supported by the holding portions is spaced from the apparatus to form a space therebetween, that is enough for the band to pass therethrough.

9. The mounting structure according to claim 1, wherein the apparatus has a trench portion recessed from an outer wall of the apparatus in which a bar part of the band fixing bar between the end portions is located.

10. The mounting structure according to claim 1, wherein the band fixing bar has a shape of a circular cylinder, a rectangular prism or a hexagonal prism.

11. The mounting structure according to claim 1, wherein:
the band fixing bar has at least one convex portion protruding from one end surface of the band fixing bar in the longitudinal direction;
a thickness of the convex portion is smaller than a thickness of the band fixing bar;
the inlet concave portion has an open shape corresponding to a sectional shape of the convex portion with the thickness; and
the convex portion is provided to pass through the inlet concave portion in a predetermined direction.

12. The mounting structure according to claim 1, wherein a maximum width between the inlet concave portions of the holding portions is greater than a dimension of the band fixing bar in the longitudinal direction.

13. The mounting structure according to claim 2, wherein the protruding portion is provided approximately in a middle area of the holding portions.

14. The mounting structure according to claim 2, wherein the protruding portion is constructed with a plurality of convex portions separate from each other in the axial direction of the band fixing bar.

15. The mounting structure according to claim 1, wherein a width of the band is greater than a dimension of the band fixing bar in the longitudinal direction.

16. A detecting apparatus for detecting a living body information, comprising:
a sensor body;
a band through which the sensor body is held to contact a surface of a living body; and
a band fixing bar elongated in a longitudinal direction, through which the band is engaged and is detachably connectable to the sensor body, wherein:
the sensor body has a pair of holding portions for holding end portions of the band fixing bar in the longitudinal direction;
the holding portion has an inlet concave portion from which the end portion of the band fixing bar is inserted, and a recess portion for receiving the end portion of the band fixing bar;
the recess portion communicates with the inlet concave portion and recesses from an end of the inlet concave portion toward the living body; and
the end portion of the band fixing bar is fitted into the recess portion.

17. The detecting apparatus according to claim 16, wherein the living body is a human body, and the sensor body is attached to detect a pulse rate of the human body.

18. The detecting apparatus according to claim 16, wherein the band fixing bar has an elasticity to be bendable in a radial direction approximately perpendicular to the longitudinal direction, the apparatus further comprising
a protruding portion located between the holding portions in the axial direction, wherein the protruding portion protrudes from the sensor body to the living body at a radial outer position of the band fixing bar with respect to the holding positions so as to prevent the band fixing bar from falling.

19. The detecting apparatus according to claim 16, wherein the recess portion of the sensor body has a lowest point on a side of the living body, the lowest point being lower than a lower end of the inlet concave portion in a state where the sensor body is positioned above the surface of the living body.

20. The detecting apparatus according to claim 16, wherein the holding portions are provided at an inner side from an outer periphery of the sensor body in the longitudinal direction.
